(19) United States
(12) Patent Application Publication
(10) Pub. No.: US 2006/0051350 A1
(43) Pub. Date: Mar. 9, 2006

(54) Methods and means to suppress symptoms of an allergic disease by inhibiting the glucocorticoid-induced tumor necrosis factor receptor (GITR or TNFRSF18)

(75) Inventors: Antonius Josephus Maria van Oosterhout, Groningen (NL); Elisabeth Catharina Adriana Maria Lobato-van Esch, Utrecht (NL); Joost Lambert Max Vissers, Nijmegen (NL)

Correspondence Address: TRASK BRITT, P.O. BOX 2550, SALT LAKE CITY, UT, 84110, US

(73) Assignee: Universiteit Utrecht Holding B.V., Utrech (NL)

(21) Appl. No.: 11/223,830

(22) Filed: Sep. 9, 2005

Related U.S. Application Data
(63) Continuation of application No. PCT/NL04/00204, filed on Mar. 25, 2004.

(30) Foreign Application Priority Data
Mar. 28, 2003 (EP) 03075908.8

(51) Int. Cl. A61K 39/395

(52) U.S. Cl. 424/133.1; 424/145.1

(57) ABSTRACT

The invention relates to the field of immunology, more in particular to the field of immune therapy, even more particularly to a method for regulating tolerance to an allergen in a subject and even more specifically, to methods which involve regulation of a glucocorticoid-induced tumor necrosis factor receptor (GITR). Provided is methods of treating allergic disorder and compositions for use therein.

### CROSS-REFERENCE TO RELATED APPLICATION

This application is a continuation of PCT International Patent Application No. PCT/NL/2004/000204, filed on Mar. 25, 2004, designating the United States of America, and published, in English, as PCT International Publication No. WO 2004/084942 A2 on Oct. 7, 2004, which itself claims priority from European Patent Application EP 03075908.8, filed on Mar. 28, 2003, the contents of the entirety of both of which are incorporated by this reference.

### TECHNICAL FIELD

The invention relates generally to biotechnology, and more particularly to the field of immunology, more in particular to the field of immune therapy, even more particularly to a method for regulating tolerance to an allergen in a subject and even more specifically, to methods which involve regulation of a glucocorticoid-induced tumor necrosis factor receptor (GITR). In one aspect, provided is methods of treating allergic disorders and compositions for use therein.

### BACKGROUND

It is now well established that regulatory T-lymphocytes can inhibit harmful immunological disorders directed against self or foreign antigens. At least three different subpopulations of regulatory T-cells have been identified: type-1 regulatory T-cells (Tr1 cells), T-helper type-3 cells (Th3 cells) and naturally occurring “professional” CD4⁺ CD25⁺ regulatory T-cells (Treg) also called suppressor cells (Roncarolo et al., 2000; Shevach, 2002).

Tr1 cells are characterized by the production of high levels of interleukin 10 (IL10) upon activation and low levels of IL2 and no IL4. Tr1 cells can be generated by repeated antigen stimulation in the presence of IL10, or vitamin D3 and dexamethasone in vitro. Generation of Tr1-like cells has also been demonstrated in human lymphocytes by repetitive stimulation (Jonuleit et al., 2000). Passive transfer of Tr1 cells has been shown to inhibit development of colitis, experimental autoimmune encephalomyelitis and Th2-mediated immune responses in mice. Tr1 cells appear to be generated in vivo by dendritic cells producing IL10, as demonstrated in a murine model of inhalation tolerance or by immature dendritic cells. Blocking of IL10 or its cell-surface receptor inhibits the immunosuppressive effects of Tr1 cells. However, cell-contact dependent immunosuppressive effects have been described as well (Jonuleit et al., 2000).

Th3 cells are characterized by the production of high levels of transforming growth factor β (TGFβ) upon activation. Th3 cells are generated by dendritic cells producing TGFβ, as demonstrated in a murine model of oral tolerance or after antigen stimulation in the presence of TGFβ. Blockade of TGFβ inhibits the immunosuppressive effects of Th3 cells on auto-immunity and transplant rejection (Roncarolo et al., 2000) as well as the immunosuppressive effects induced after oral antigen administration (so-called “oral tolerance”).

Naturally occurring “professional” CD4⁺ CD25⁺ regulatory T-cells (Treg cells), also called suppressor cells (the terms will be used interchangeably herein), are generated in the thymus and are present in the blood and peripheral tissues at a frequency of approximately 5-10% of all peripheral CD4⁺ cells (Roncarolo et al., 2000; Shevach, 2002). It is now generally accepted that CD4⁺ CD25⁺ Treg cells play an important role in the induction and maintenance of peripheral self-tolerance. Passive transfer of these lymphocytes has been shown to suppress autoimmune diabetes, inflammatory bowel disease and transplant rejection in rodents (Roncarolo et al., 2000). Treg cells require activation via the T-cell receptor to exert their regulatory function, however, once activated, their suppressive effects are antigen non-specific indicating that these cells mediate bystander suppression in vivo (Thornton et al., 2000).

In humans, CD4⁺ CD25⁺ Treg cells have also been described (Jonuleit et al., 2001 a). All of the functional studies with human CD4⁺ CD25⁺ Treg cells confirm what has been described with murine T-cells. They show strongly reduced proliferative responses upon stimulation even in the presence of IL-2 and suppress proliferative responses of CD4⁺ CD25⁺ T-cells. Both human and murine CD4⁺ CD25⁺ Treg cells are able to produce low levels of the immunosuppressive cytokines IL10 and TGFβ. However, suppression cannot be overcome by addition of neutralizing antibodies to these cytokines or their respective cell-surface receptors (Jonuleit et al., 2001 a). In contrast to Th3 and Tr1 cells, it has been demonstrated that immune suppression by CD4⁺ CD25⁺ “professional” Treg cells is mediated by a cell-contact dependent mechanism (Shevach, 2002). Although naturally occurring Treg cells are thought to be generated in the thymus, other ways to induce these cells appear to exist. Jonuleit and colleagues (Jonuleit et al., 2000) demonstrated that repetitive stimulation of naive T-cells with allogeneic immature dendritic cells induces a population of T cells that resemble naturally occurring CD4⁺ CD25⁺ Treg cells since their suppressive phenotype was dependent on cell-contact, antigen non-specific and APC independent.

The expression of CD4 and CD25 by Treg cells cannot completely discriminate between Treg cells and activated T-cells. Although not exclusively expressed on Treg cells, other markers have been used to identify these cells such as CTLA4, CD45RB/CD45RO (mouse/human) and glucocorticoid-induced tumor necrosis factor receptor (GITR).

Glucocorticoid-induced TNF receptor (GITR, also known as TNFRSF 18) was recently identified by differential gene expression (McHugh et al., 2002). Ligation of GITR by an activating antibody appeared to inhibit the suppressive effect of Treg cells on T-lymphocyte activation in vitro (McHugh et al., 2002; Shimizu et al., 2002). GITR can be ligated by its natural (or endogenous, the terms will be used interchangeably herein) ligand, GITR ligand (GITRL), leading to the activation of the NF-κB pathway in T-cells and prevention of apoptosis. GITRL is a member of the TNF family and encodes a 177 amino-acid polypeptide with a calculated mass of 20 kDa, and with a type 2 transmembrane topology.

The role of each of the different regulatory T-cell subsets (Th3, Tr1, Treg) in allergic diseases is at present largely unknown. In a mouse model of asthma, it has been shown that treatment with heat-killed Mycobacterium vaccae can suppress airway eosinophilia which appears to be mediated by CD4⁺ CD45RB^{lo} T-cells (Zuany-Amorim et al., 2002). Anti-IL-10 and anti-TGFβ inhibit these suppressive effects of M. vaccae, pointing to a role for both Tr1 and Th3 cells but not to a role for naturally occurring Treg cells. In a model of inhalation tolerance, it has been shown that the prevention of asthma manifestations in a mouse model was mediated by IL-10 producing Tr1 cells. Furthermore, we have demonstrated that the beneficial effects of allergen-immunotherapy in a mouse asthma model are mediated by IL-10, pointing to a role for Tr1 cells in allergen immunotherapy. Similarly, bee-venom immunotherapy in humans has been shown to be mediated by IL-10 producing lymphocytes. Suto and colleagues (Suto et al., 2001) have shown that naturally occurring CD4⁺ CD25⁺ Treg cells potentiate Th2 responses and inhibited Th1 responses in a model of antigen-induced eosinophil recruitment after transfer of ovalbumin-specific TCR transgenic T-cells.

Murine models of allergic asthma have widely been accepted as a suitable animal model to study the mechanisms underlying this disease. Previously, we developed a mouse model with immunologic and pathophysiologic features reminiscent of allergic asthma. In this model, Balb/c mice are sensitized with ovalbumin and repeatedly challenged by inhalation of ovalbumin aerosol. This model is characterized by the presence of ovalbumin-specific IgE antibodies in serum, airway eosinophilia and non-specific hyper responsiveness concomitant with the appearance of Th2-like cells in lung tissue and lung draining (thoracic) lymph nodes. The role of T-helper lymphocytes in these allergic symptoms has been clearly demonstrated by depletion of CD4⁺ T-cells or a specific T-cell subset as well as by transfer of antigen-specific Th2 cells. In addition, several Th2 derived cytokines including IL4, IL5, IL9 and IL13 are implicated in one or more of these asthma-like symptoms whereas treatment with the Th1 derived cytokine IFNγ down-regulates these characteristics. Hence, Th2-lymphocytes play a role in the initiation and progression of allergic asthma in this mouse model.

At present, glucocorticoids are generally considered the most effective drugs in the treatment of asthma to reduce the inflammatory component and hyper responsiveness. However, glucocorticoids are not very selective and affect a whole range of inflammatory and non-inflammatory cells. Thus, there is a strong need for novel (possibly combinatorial) therapeutic strategies that are more selective and induce long-term relief of symptoms.

### SUMMARY OF THE INVENTION

Disclosed is that the number of naturally occurring CD4⁺ CD25⁺ Treg cells, upon allergen challenge, significantly increases in the lung tissue but not in the draining lymph nodes in a mouse model of allergic asthma (Example 1). Moreover, these Treg cells suppress the allergen induced airway hyper responsiveness (AHR) as demonstrated by the strong potentiation of AHR upon inactivation of Treg cells by ligation of GITR using an activating monoclonal antibody (Example 2). In addition, up-regulation of serum IgE levels after allergen challenge is strongly potentiated upon inactivation of Treg cells (Example 2). The up-regulation of AHR and serum IgE levels after treatment with anti-GITR coincides with increased production of Th2 cytokines upon restimulation in vitro (Example 3).

These data demonstrate that allergen inhalation stimulates the recruitment of Treg cells, preferably at a site where symptoms associated with an allergic disease are present, and that these cells are able to down-regulate the symptoms. In one aspect, provided is methods to increase the suppressor activity and/or number of CD4⁺ CD25⁺ Treg cells, which are useful for the treatment of allergic disorders for example, asthma. It is clear that such methods are also applicable in the treatment of related immune- or inflammatory disorders, including autoimmune diseases and transplant rejection.

In a first embodiment, provided is a method for regulating tolerance to an allergen in a subject, comprising providing, in the presence of the allergen, the subject with an inhibitor capable of regulating the activation of a glucocorticoid-induced tumor necrosis factor receptor (GITR). In yet another embodiment, provided is a method for inducing and/or increasing tolerance to an allergen in a subject, comprising providing, in the presence of the allergen, the subject with an inhibitor capable of at least in part preventing activation of GITR. Or in other words, provided is a method to at least in part diminish (or more preferably completely diminish) symptoms associated with the presence of an allergen and thus to diminish symptoms of an allergic disease.

An allergen is typically defined as a compound that is capable of inducing and/or maintaining symptoms indicative of an allergic disease. Typical examples of allergens are dust, pollen, grasses, some medications, proteins, and metals.

Examples of allergic diseases, which can be treated according to a method of the invention include, but not limited to allergic rhinitis, food allergy, urticaria, atopic dermatitis, allergic conjunctivis, angioedema, asthma, and insect sting allergy.

As one of the main representatives of the family of allergic diseases, we will describe asthma in greater detail as representative of the applications of the invention. Allergic asthma is, amongst other things, characterized by reversible airway obstruction, elevated levels of IgE, chronic airway inflammation and airway hyper responsiveness to bronchospasmogenic stimuli, airway tissue remodeling and mucus hyper secretion. The allergic inflammatory infiltrate in the airway tissue predominantly consists of eosinophils and CD4⁺ T-lymphocytes. It is now widely accepted that type 2 T-helper (Th2) lymphocytes, which produce a limited set of cytokines including interleukin-3 (IL3), IL4, IL5, IL9, IL10 and IL13 play a role in the initiation and progression of allergic asthma.

As disclosed herein in the experimental part, the number of naturally occurring CD4⁺ CD25⁺ Treg cells, upon allergen challenge, significantly increases in the lung tissue but not in the draining lymph nodes in a mouse model of allergic asthma. Moreover, these Treg cells suppress the allergen induced airway hyper responsiveness (AHR) as demonstrated by the strong potentiation of AHR upon inactivation of Treg cells by ligation of GITR using an activating monoclonal antibody. In addition, up-regulation of serum IgE levels after allergen challenge is strongly potentiated upon inactivation of Treg cells. The up-regulation of AHR and serum IgE levels after treatment with anti-GITR coincides with increased production of Th2 cytokines upon restimulation in vitro. Hence, by providing an inhibitor capable of at least in part preventing the activation of GITR to a subject in whom an allergen (which presence results in symptoms of an allergic reaction/disease) is present, symptoms associated with the disease are diminished. More in specific for asthma, a method according to the invention results in down regulation of Th2-lymphocyte activation and cytokine production leading to suppression of allergen-induced AHR and serum IgE levels and to at least in partial diminishing of symptoms associated with the presence of the allergen in the subject and tolerance to the allergen is induced or increased.

Preferably, the subject is a mammal and even more preferably the mammal is a human being. Hence, provided is a method to treat an allergic disease/disorder in a human being. A human being suffering from the symptoms induced and/or maintained by an allergen (and hence suffering from an allergic disease) is now treated by providing the human being with an inhibitor capable of at least in part preventing/blocking of GITR activation and this blocking leads to functional disengagement of Treg cells and the subsequent suppression of Th2-mediated allergic disorder.

### DESCRIPTION OF FIGURES

FIG. 1. Airway responsiveness to inhalation of different doses of methacholine. Ovalbumin sensitized BALB/c mice (n=6 per group) were treated with control antibody (open and hatched bars) or anti-GITR (black and cross-hatched bars) prior repeated ovalbumin inhalation challenges. Airway responsiveness was measured before (black and white bars) and after (hatched and crosshatched bars) ovalbumin inhalation challenges. The methacholine dose-response curves after ovalbumin challenge are significantly (P<0.01) different from those before challenge. The methacholine dose-response curve after ovalbumin challenge of mice treated with anti-GITR is significantly (P<0.01) different from the curve after ovalbumin challenge of mice treated with control antibody.

FIG. 2. Production of the prototypic Th2 cytokine IL-5 by lung lymphocytes derived from OVA challenged mice cultured for 5 days with medium only (cntr) or restimulated with plate-bound anti-CD3 (aCD3) or ovalbumin (OVA, 10 μg/ml) in the presence of A: control antibody (n=3, white bars) or 10 μg/ml anti-GITR (n=3, black bars) or B: control IgG (n=3, white bars) or 1 μg/ml GITR-Fc (n=3, black bars).
**: P<0.01 as compared to control antibody; *: P<0.05 as compared to control antibody.

FIG. 3. Airway responsiveness to inhalation of different doses of methacholine was measured before (pre, last 4 bars) and after (after, first 4 bars) OVA inhalation challenge. Ovalbumin sensitized BALB/c mice (n=4 to 6 per group) were sham-treated (sham) or received OVA-immunotherapy (IT). Prior to immunotherapy, mice were injected with 1 mg anti-GITR (aGITR) or control antibody (cntr). After OVA-inhalation challenge, all mice display significant airway hyperreactivity to methacholine as compared to before challenge.
*: P<0.05 as compared to sham-treatment; #: P<0.05 as compared to control antibody treated mice.

FIG. 4. Number of leukocytes in BALF after OVA inhalation challenge of mice injected with control antibody (cntr) or anti-GITR (aGITR) prior to sham-immunotherapy (sham) or OVA-immunotherapy (IT). MNC: mononuclear cells; EO: eosinophils; NEUTRO: neutrophils; TCC: total cell counts.
*: P<0.05 as compared to sham-treated group; #:P<0.05 as compared to control antibody treated mice.

### BEST MODE OF THE INVENTION

The term “at least in part preventing activation of GITR” is defined herein in that the symptoms associated with an allergic disease are at least in part diminished, more preferably complete inhibited, preferably resulting at least in an improvement of overall well being.

Preferably, the inhibitor at least in part prevents ligation of GITR by an endogenous GITR-ligand (GITRL). However, now that it is clear that Treg cells (that comprise the GITR) play such an important role in the induction and/or maintenance of an allergic disease it is also well within the scope of the present invention to provide an inhibitor that is capable of inhibiting the downstream signaling pathway of GITR, for example by interfering in the NF-κB pathway. Examples of NF-κB inhibitors are glucocorticoids and non-steroidal anti-inflammatory drugs such as aspirin and cAMP elevating compounds such as beta-adrenoceptor agonists.

Examples of inhibitors that are capable of (at least in part) preventing ligation of GITR by an endogenous GITRL are an inhibitor of GITR or an inhibitor of GITRL or a compound that at least in part inhibits the expression of GITR or GITRL.

An inhibitor of GITR is typically capable of binding to GITR without activating the receptor and hence such a compound blocks binding of an endogenous GITRL to GITR and hence activation of GITR is prevented, resulting in the presence of immunosuppressive Treg cells. Examples of these kinds of compounds are antagonists of GITR including but not limited to non-activating antibodies or antibody fragments, peptides or (small) synthetic molecules.

An inhibitor of GITRL is typically capable of binding to GITRL in such a way that further binding to GITR is blocked and hence a similar result as described for the inhibitor of GITR is obtained. Examples of these kinds of compounds include, but are not limited to chimaeric fusion proteins such as GITR-Fc or antibodies or antibody fragments, peptides or (small) synthetic molecules.

Yet another way to interfere with ligation of GITR by an endogenous GITRL is by interfering with the expression of either GITR or GITRL. When the endogenous GITR ligand is not expressed, binding with GITR will not take place, resulting in immunosuppressive Treg cells and an induction and/or increase in tolerance to an antigen is obtained. Examples of such inhibitors are anti-sense oligonucleotides or small interfering RNA molecules.

Depending on the nature of an inhibitor that is capable of at least in part preventing activation of GITR, the inhibitor can be administered either enterally, parenterally or preferably targeted at the site where the symptoms due to the presence of an allergen are present, i.e., inhalation in case of asthma using targeting devices such as liposomes. Hence, the inhibitor is preferentially provided locally.

In a preferred embodiment, provided is a method for inducing and/or increasing tolerance to an allergen in a subject, comprising providing, in the presence of the allergen, the subject with an inhibitor capable of at least in part preventing activation of GITR, wherein the subject is further provided with the allergen. It is clear that it is possible that the allergen is already present in the subject, for example because the allergen (for example pollen) has entered the subject via inhalation. However, it is also possible that an allergen responsible for the induction and/or maintenance of an allergic disease is temporarily not present or no longer present or is present in a too low concentration. In these circumstances it is particularly advantageous to provide the subject with the inhibitor capable of at least in part preventing activation of GITR in combination with the allergen. It is clear that the inhibitor and the allergen can be provided to the subject in the same or different compositions, sequentially or at the same time. A sequential combination can either be first an inhibitor followed by an allergen or first an allergen followed by an inhibitor.

CD4⁺ CD25⁺ Treg cells can induce long-lasting anergy and production of IL10 or TGFβ in CD4⁺ CD25 (Dieckmann et al., 2002; Jonuleit et al., 2002). The immunosuppressive effects of these T-cells depends on IL10 or TGFβ and can therefore be classified as Tr1 or Th3 cells. Thus, methods to enhance the immunosuppressive effects of Treg cells as described above can be used in combination with antigen (allergen, auto-antigen, allo-antigen) to induce the differentiation of antigen-specific Tr1 and/or Th3 cells. This strategy leads to even stronger suppression of Th1- or Th2 mediated immune responses. Moreover, induction of antigen-specific Tr1 cells will lead to long-term immunosuppressive effects in an antigen-specific fashion. Preferably, the allergen is an allergen involved in the induction and/or maintenance of an allergic disease, especially asthma and hence provided is a method for inducing and/or increasing tolerance to an allergen involved in asthma. Allergens for use in a method according to the invention include, but are not limited to the list available on the World-Wide Web at www.allergen.org/List.htm. Typical example of allergens that result in asthma symptoms are small particles, like house-dust or pollen.

In yet another embodiment, provided is a method for inducing and/or increasing tolerance to an allergen in a subject, comprising providing, in the presence of the allergen, the subject with an inhibitor capable of at least in part preventing activation of GITR, wherein the method further comprises providing the subject with a compound capable of activating a Toll-like receptor. Preferably, the Toll-like receptor (TLR) is TLR4 and/or 5 and/or 7 and/or 8. The immunosuppressive effects of CD4⁺ CD25⁺ Treg cells are further potentiated by activation of specific Toll-like receptors. CD4⁺ CD25⁺ Treg cells in normal naive mice have been shown to selectively express TLR4, 5, 7 and 8, whereas TLR1, 2 and 6 are more broadly expressed on all CD4⁺ T cells (Caramalho et al., 2003). Different TLRs are activated by different microbial products. Ligation of TLR-4 by the ligand lipopolysaccharide strongly increased the suppressor activity of Treg cells (Caramalho et al., 2003). However, TLRs are also expressed on APCs and ligation of these receptors can trigger their maturation, which can enhance the induction and activation of Th1 or Th2 responses. Moreover, the suppressor function of Treg cells can be overridden by activation TLR4 or TLR9 ligands on APCs leading to the production of IL-6 (Pasare et al., 2003). Therefore, targeting of selective TLR ligands (in particular ligands for TLR4, 5, 7 and 8) to Treg cells but not to APCs can be used to increase the immunosuppressive effects of Treg cells.

In another embodiment, provided is a method for inducing and/or increasing tolerance to an allergen in a subject, comprising providing, in the presence of the allergen, the subject with an inhibitor capable of at least in part preventing activation of GITR, further providing the subject with a compound capable of inhibiting activation of an antigen presenting cell. Another strategy to selectively stimulate Treg function by TLR ligands without potentiating APC function is the inhibition of APC maturation by a compound that inhibits NF-κB and/or MAPK signal transduction pathways or by blocking of IL-6, its production, or its cell-surface receptor at the time of treatment with TLR ligands. The NF-κB transducing pathway can, for example, be inhibited by a compound chosen from the group of anti-oxidant compounds and/or proteasome and/or protease inhibitors, IKB phosphorylation and/or degradation inhibitors, and/or a functional analogue thereof, or by a compound chosen from the group of anti-inflammatory compounds, and/or by a compound chosen from the group of glucocorticosteroid compounds, and/or by a compound chosen from the group of di-hydroxyvitamin D3 compounds and/or a functional analogue thereof, or by a compound from the group of cAMP elevating compounds, and/or by NF-KB decoy- or anti-sense oligonucleotides. The MAPK/AP-1 signal transducing pathway can for example be inhibited by a compound chosen from the group of non-steroidal anti-inflammatory compounds and/or a functional analogue thereof, and/or by a ligand to a peroxisome proliferator-activated receptor and/or a functional analogue thereof, and/or by a compound of the group of pyridinylimidazole compounds and/or a functional analogue thereof, and/or AP-1 decoy- or anti-sense oligonucleotides.

However, it is also possible to further potentiate the immunosuppressive effects of CD4⁺ CD25⁺ Treg cells by activation of the cell-surface receptor CTLA4, which is constitutively expressed by these cells but not by resting CD4⁺ CD25⁺ T-lymphocytes. CTLA4 is a member of the T-cell costimulatory molecules that can provide a negative signal for T-cell activation. The endogenous ligands for CTLA4 are CD80 and CD86 that can be expressed on antigen-presenting cells, in particular mature dendritic cells. However, CD28 that is constitutively expressed by all T-cells is also ligated by CD80 and CD86, leading to a co-stimulatory signal for T-cells activation upon simultaneous stimulation through their T cell receptor. Since CTLA4 is constitutively present on CD4⁺ CD25⁺ Treg cells but not on resting CD4⁺ CD25⁺ T cells, ligands that activate this receptor selectively potentiate the immunosuppressive function of Treg cells. Activation of CTLA4 is for example accomplished by a selective monoclonal antibody that is able to activate the receptor and its down-stream signaling pathway. In addition, compounds that have a selective agonistic effect on CTLA4 can also be used for this purpose.

At present, glucocorticoids are used in the treatment of asthma. There is a tendency to at least decrease the amount of used glucocorticoids or preferably, due to its non-selective action, to find an alternative treatment. The invention also provides a method to reduce the amount of GITR in a treatment of allergic diseases. GITR has been discovered by treatment of a mouse T-cell hybridoma with glucocorticoids. Interestingly, glucocorticoids are widely used for the treatment of allergies and asthma. It is anticipated that glucocorticoids interfere with the protective role of naturally occurring Treg cells by increasing GITR expression and hence making these cells more sensitive to inhibition by GITRL. Treatment with inhibitors that inhibit the activation of GITR or its down-stream signaling pathway act synergistically with glucocorticoids for the treatment of allergic diseases, including asthma and other chronic inflammatory diseases such as auto-immune diseases. A strong need exists for so-called “add-on” therapies to limit the use of high doses of glucocorticoids and the associated side effects. The present invention provides with a combination treatment of glucocorticoids and a compound capable of at least in part preventing activation of GITR, such an “add-on” treatment.

In yet another embodiment, provided is a method for obtaining a compound capable of at least in part preventing activation of GITR, the method comprising the steps of
incubating a GITR protein or a functional equivalent and/or a functional fragment thereof with a candidate compound
determining whether the candidate compound binds to the GITR protein or a functional equivalent and/or a functional fragment thereof
determining whether the candidate compound blocks or mimics an effect mediated by a GITR-GITRL interaction
selecting a compound that blocks an effect mediated by a GITR-GITRL interaction.

In a preferred embodiment, the method further comprises testing the compound in a non-human animal with features associated with an allergic disease. Even more preferably, the allergic disease is asthma and the non-human animal is a mouse.

A GITR protein or a functional equivalent and/or a functional fragment thereof is for example obtained by isolation of GITR protein from cells naturally expressing GITR protein or from cells (mammalian or bacterial or insect) transfected with GITR cDNA and expressing GITR protein. A functional equivalent and/or a functional fragment of a GITR protein is a protein which is capable of performing essentially the same function as the GITR protein, but not necessarily in the same amount. Typically, the equivalent and/or fragment is capable of binding an (endogenous) GITRL and preferably also capable of activating the downstream signaling pathway. Besides using (partially) purified GITR protein it is also possible to provide the GITR protein as a (cell) membrane-GITR protein complex.

GITR protein or the active part involved in binding to GITRL or GITR expressing cells or cell-membrane preparations derived from these cells are used for screening compounds that bind selectively and with high affinity to this receptor. Examples of such compounds are antibodies or functional fragments thereof, variants (one or more amino acid substitutions) of GITRL or variants of the part involved in binding to GITR, peptides based on GITRL pharmacophore or compounds can be present in random or focused compound libraries. Screening is, for instance, carried out using so-called competition- or displacement binding assays consisting of solubilized GITR protein or the active part involved in binding to GITRL or GITR expressing cells or cell-membrane preparations derived from these cells and a constant amount of labeled (radioactive, fluorescent) ligand (i.e., GITRL or the part involved in binding to GITR). Compounds that bind with sufficient affinity to GITR will compete with ligand binding to GITR and are lead inhibitors. To determine the binding affinity of the lead inhibitor to GITR, several technologies are available such as (radio-) ligand receptor binding assays or scintillation proximity assay or surface plasmon resonance.

Compounds with sufficient affinity for GITR are then, for example, screened in cell-based functional assays whether they block (antagonist) or mimic (agonist) a biological effect mediated by GITR:GITRL interaction in a cell expressing GITR protein. Primary cells expressing GITR isolated from humans or animals or a cell-line expressing GITR or a transfected cell-line (e.g., HEK293) expressing GITR and responding to the activation of GITR by an agonist (e.g., an activating antibody, GITRL) may be used to determine the (ant)agonistic capacity of the compounds. Whether a cell is responding is determined directly by measuring a relevant (biological) response such as the activation of the signal transduction pathway (leading to NF-KB activation) or protection from apoptosis or by using a reporter gene (i.e., luciferase) construct or indirectly by using a bioassay with cells of which the biological function (i.e., proliferation) is sensitive to inhibition by the GITR expressing cell.

Compounds that are capable to block the activation of GITR by GITRL are further screened in a non-human animal model with features associated with an allergic disease or in a non-human animal model in which naturally occurring Treg cells are involved to test their in vivo capability.

It is clear that this method for obtaining a compound capable of at least in part preventing activation of GITR, can also be based on GITRL. The method comprises the steps of
incubating a GITRL protein or a functional equivalent and/or a functional fragment thereof with a candidate compound
determining whether the candidate compound binds to the GITRL protein or a functional equivalent and/or a functional fragment thereof
determining whether the candidate compound blocks or mimics an effect mediated by a GITR-GITRL interaction
selecting a compound that blocks an effect mediated by a GITR-GITRL interaction.

In a further embodiment, provided is an isolated, recombinant or synthetic compound obtainable according to the method of the invention. Preferably, the isolated, recombinant or synthetic compound is a proteinaceous substance, like an antibody or a functional equivalent and/or a functional fragment thereof or a peptide.

In yet another embodiment, provided is a nucleic acid encoding a proteinaceous compound capable of at least in part preventing activation of GITR. In a preferred embodiment the nucleic acid is part of a vector and more preferably provided is a gene delivery vehicle comprising the nucleic acid or the vector.

By equipping a gene delivery vehicle with a nucleic acid molecule encoding a compound capable of at least in part preventing activation of GITR and by targeting the vehicle in a subject to a site where symptoms associated with the presence of an allergen are present, the gene delivery vehicle provides the subject with the necessary means of preventing activation of GITR and to subsequent suppression of Th2-mediated allergic disorders. Such a gene delivery vehicle, which is an independently infectious vector may for example be a virus (like an adenovirus or a retrovirus), or a liposome, or a polymer, or the like, that in it self can infect or in any other way deliver genetic information to a preferred site of treatment. Furthermore, provided is a gene delivery vehicle which has additionally been supplemented with a specific ligand or target molecule or target molecules, by which the gene delivery vehicle can be specifically directed to deliver its genetic information at a target cell of choice. Such a target molecule may for instance be a receptor molecule.

In yet another embodiment, provided is a pharmaceutical composition comprising an isolated, recombinant or synthetic compound capable of at least in part preventing activation of GITR, a nucleic acid encoding a proteinaceous compound, a vector or a gene delivery vehicle.

A pharmaceutical composition can either be in a solid form (for example, a pill, tablet, or capsule) or in a fluidised form (for example, a liquid formulation). It is clear to a person skilled in the art that the active ingredient (for example, a proteinaceous substance capable of at least in part preventing activation of GITR) can be accompanied by a pharmaceutical acceptable carrier or diluent. Such a pharmaceutical can be applied via different routes, for example via a local injection, dermal application, intravenously or via inhalation. It is clear to a person skilled in the art that the pharmaceutical can further comprise a compound capable of activating a Toll-like receptor and/or a compound capable of inhibiting activation of an antigen presenting cell and/or a compound that has a selective agonistic effect on CTLA4. In a preferred embodiment, a pharmaceutical composition according to the invention further comprises an allergen, more preferably the allergen is an allergen involved in allergic disease and even more preferably the allergic disease is asthma.

The invention furthermore provides use of an isolated, recombinant or synthetic compound capable of at least in part preventing activation of GITR, a nucleic acid encoding the proteinaceous compound, a vector or a gene delivery vehicle for the preparation of a medicament for the treatment of an allergy, for example asthma. Preferably, the compound is an inhibitor of GITR or the compound at least in part inhibits the expression of GITR or the compound is an inhibitor of a GITRL or the compound at least in part inhibits the expression of GITRL.

In yet another embodiment, provided is a method for inducing and/or increasing tolerance to an allergen in a subject, comprising increasing in the subject in which the allergen is present the number of CD4⁺ CD25⁺ GITR+T cells at a site of allergic disease. The present invention discloses the immunosuppressive function of Treg cells with regard to symptoms induced and/or maintained by an allergen and hence increasing the number of Treg cells (CD4⁺ CD25⁺ GITR+T cells) results in an increase in immune suppression. To increase the number of Treg cells at site of immune- or inflammatory responses, local administration of chemokines that are involved in the recruitment of Treg cells are used. Chemokines and adhesion molecules regulate the migration of functionally different T-cells to lymphoid- or peripheral tissues. CD4⁺ CD25⁺ Treg cells respond to several inflammatory and lymphoid chemokines in particular the chemokines CCL1 (1-309), CCL4 (MIP-1β), CCL17 (TARC), CCL22 (MDC) (Bystry et al., 2001; Colantonio et al., 2002; lellem et al., 2001). In agreement herewith, CD4⁺ CD25⁺ Treg cells specifically express the chemokine receptors CCR4, CCR5 and CCR8 (Bystry et al., 2001; lellem et al., 2001). Importantly, APCs can produce these kind of chemokines upon activation or maturation and subsequently can attract CD4⁺ CD25⁺ Treg cells to sites of inflammation or immune responses (Bystry et al., 2001; lellem et al., 2001). However, these pathways for recruitment to sites of inflammation are not unique for Treg cells since these receptors are also expressed by Th1 and Th2 lymphocytes. Thus, to increase the number of Treg cells in Th1-dominated inflammatory responses, local administration (i.e., affected joint in case of arthritis) of chemokines that activate CCR8 and/or CCR4 will be effective. This may be accomplished by agonists including but not limited to 1-309, TARC and MDC or functional analogues (small molecules, peptides) of these chemokines. To increase the number of Treg cells in Th2-dominated inflammatory responses, local administration (i.e., inhalation in case of asthma) of chemokines that activate CCR5 will be effective. This may be accomplished by agonists including but not limited to MIP-1β, MIP-1α, RANTES or functional analogues (small molecules, peptides) of these chemokines.

Preferably, the increase in the number of Treg cells is obtained by providing the subject with chemokines or a functional fragment and/or a functional analogue thereof and even more preferably the chemokines are locally administered. Depending on which T cells are involved the immune response to an allergen; the following options can be applied. When the allergic response is a Th1-dominated response, the chemokine or a functional fragment and/or a functional analogue thereof is capable of activating CCR8 and/or CCR4. Examples of chemokines that can be applied in a Th1-dominated response are 1-309, TARC or MDC or a functional fragment and/or a functional analogue thereof. However, when the allergic response is a Th2-dominated response, the chemokine or a functional fragment and/or a functional analogue thereof is capable of activating CCR5. Examples of chemokines that can be applied in a Th2-dominated response are MIP-1β, MIP-1α or RANTES or a functional fragment and/or a functional analogue thereof.

The method that involves increasing the number of Treg cells can be combined with another method, as described herein, for increasing the immunosuppressive capability of Treg cells, for example by combining with a compound capable of at least in part preventing activation of GITR and/or a compound capable of activating a Toll-like receptor and/or a compound capable of inhibiting activation of an antigen presenting cell and/or a compound capable of activating CTLA4.

In yet another embodiment, provided is a method for suppressing GITR activation of cells expressing the GITR, comprising providing, in the presence of an allergen, the cells with an inhibitor of GITR. The method is easily performed in vitro, and hence the method is preferably used in screening assays.

In a preferred embodiment, GITR activation of GITR expressing cells can be achieved by providing the cells with an inhibitor of GITR which comprises GITR-Fc. The GITR-Fc binds to GITRL and prevents the binding if GITRL to GITR.

In another embodiment, disclosed is a method for selecting an allergic individual responsive to allergen-immune treatment (also called allergen-immunotherapy or specific immunotherapy), comprising measuring whether the number of CD4⁺ CD25⁺ Treg cells in the blood of the individual is sufficient to start the treatment in the allergic individual.

In yet another embodiment, disclosed is a method for determining responsiveness of an allergic individual to allergen-immune treatment (also called allergen-immunotherapy or specific immunotherapy), comprising measuring whether the number of CD4⁺ CD25⁺ Treg cells in the blood of the individual increases as a result if the treatment.

Increasing the number and/or function of natural Treg cells in an individual will increase the tolerance to allergens, therefore, the present invention provides a method to improve tolerance to an allergen in an individual comprising increasing the number and/or function of natural Treg cells in the individual. Further, having increased numbers of Treg cells in the body enables a person to respond better to allergens, meaning that not an allergic response is elicited but a tolerogenic response. Therefore, increasing the number of Treg cells before, or during exposure to an allergen decreases the allergic reaction to the allergen. Therefore, disclosed is a method to improve tolerance to an allergen comprising increasing the number and/or function of natural Treg cells in the individual, wherein the increasing is prior to, or concomitant with providing the allergen. In a preferred embodiment, the above described methods contact T-cells with TGFbeta to cause the increase of the Treg numbers. Therefore, disclosed is a method as described above, wherein the increasing is caused by contacting T-cells with TGFbeta.

The invention will be explained in more detail in the following illustrative Examples, which are not limiting the invention.

### Experimental Part 1

### Materials and Methods

Animals. Animal care and use were performed in accordance with the guidelines of the Dutch Committee of Animal Experiments. Specific pathogen-free male BALB/c mice (6-8 wk old) were purchased from Charles River (Maastricht, NL) and housed in macrolon cages in a laminar flow cabinet and provided with food and water ad libitum.

Sensitization, treatment and challenge. Mice were sensitized intraperitoneally (i.p.) on days 0 and 7 with 10 μg OVA (grade V, Sigma-Aldrich) in 0.1 ml alum (Pierce, Rockford, Ill.). After two weeks, sensitized mice were exposed to three OVA inhalation challenges (10 mg/ml in saline) for 20 min every third day. In some experiments, mice were treated by i.p. injection of 1 mg rat monoclonal antibody to mouse GITR (DTA-1) or control antibody (rat IgG) prior to OVA inhalation challenge.

Isolation of lung cells for flow cytometry and restimulation in vitro. Mice were sacrificed by i.p. injection of 1 ml 10% urethane (Sigma-Aldrich) in saline and lungs were perfused via the right ventricle with 4 ml saline containing 100 U/ml heparin (Leo Pharmaceuticals, Weesp, NL). Lungs were perfused via the right ventricle with 4 ml saline containing 100 U/ml heparin (Leo Pharmaceuticals). Next, the thoracic lymph nodes (TLN), derived from the paratracheal and parabronchial regions, and the lungs were removed. Subsequently, single-cell suspension from lungs and TLNs were incubated with Ab against FcyRII/III (24G₂) to block aspecific binding. To determine the number of CD4⁺ CD25⁺ CD45RB^{lo} Treg cells, cell suspensions were stained with Cy-Chrome™-conjugated anti-CD4 (clone H129.19), PE-conjugated anti-CD25 (clone PC61) and FITC-conjugated anti-CD45RB (clone 16A). After staining, cells were analyzed with FACScan™ flow cytometer (Becton Dickinson) using the CELLQuest™ software program.

For T-lymphocyte restimulation, lung cells were suspended in culture medium (RPMI 1640 enriched with 10% FCS, 1% glutamax I, gentamicin (all from Life Technologies, Gaithersburg, Md.), and 50 μM P-mercaptoethanol (Sigma-Aldrich, Zwijndrecht, NL)) at a concentration of 6×10⁵ cells/well in round-bottom 96-well plates (Greiner Bio-One GmbH, Kremsmuenster, Austria) in the absence or presence of plate-bound rat anti-mouse CD3 mAb (clone 17A2, 50 μg/ml, coated 16 h at 4° C.). Each stimulation was performed in triplicate. After 5 days of culture at 37° C., the supernatants were harvested, pooled per stimulation, and stored at −20° C. until cytokine levels were determined by ELISA.

Cytokine ELISAs. IL-5, IL-10 and IL-13 (BD PharMingen) ELISAs were performed according to the manufacturer's instructions. The detection limits of the ELISAs were 32 pg/ml for IL-5, 15 pg/ml for IL-10 and 15 pg/ml for IL-13.

Measurement of airway responsiveness in vivo. Twenty-four hours after the last OVA challenge, airway responsiveness was measured in conscious, unrestrained mice using barometric whole-body plethysmography by recording respiratory pressure curves (Buxco, EMKA Technologies, Paris, FR) in response to inhaled methacholine (acetyl-p-methylcholine chloride, Sigma-Aldrich). Airway responsiveness was expressed in enhanced pause (Penh), as described in detail previously (Deurloo et al., 2001). Briefly, mice were placed in a whole-body chamber and basal readings were determined for 3 min. Aerosolized saline, followed by doubling concentrations of methacholine (ranging from 1.6-25 mg/ml in saline), were nebulized for 3 min, and readings were determined for 3 min after each nebulization. The effective dose of methacholine that induced a half-maximal response, the ED₅₀ value, was calculated after correction for baseline Penh values.

Determination of OVA-specific immunoglobulin levels in serum. After measurement of airway responsiveness in vivo, mice were sacrificed by i.p. injection of 1 ml 10% urethane in saline and were bled by cardiac puncture. Subsequently, serum was collected and stored at −70° C. until analysis. Serum levels of OVA-specific IgE were measured by sandwich ELISA as described previously (Deurloo et al., 2001).

Statistical analysis. All data are expressed as mean±standard error of mean (SEM). The airway dose-response curves to methacholine were statistically analyzed by a general linear model of repeated measurements followed by post-hoc comparison between groups. Data were log transformed before analysis to equalize variances in all groups. All other data were analyzed using a Student's t test (2-tailed, homosedastic). Results were considered statistically significant at the p<0.05 level.

Results

### EXAMPLE 1

The Number of Naturally Occurring Professional Treg Cells is Increased in Lungs Obtained from a Mouse Model of Allergic Asthma

We examined whether the number of naturally occurring professional Treg cells changes upon allergen inhalation in a mouse model of allergic asthma. Balb/c mice were sensitized by the model allergen ovalbumin in the presence of the adjuvant alum as described previously (Deurloo et al., 2001). Upon repeated allergen inhalation challenge, mice develop airway manifestations of allergic asthma such as airway hyperresponsiveness to methacholine and eosinophilic airway inflammation (Deurloo et al., 2001). To determine the number of professional Treg cells, lung tissue cells and lung-draining lymph node cells were isolated 24 hours after the last challenge, as described previously (Deurloo et al., 2001) and stained using monoclonal antibodies to specific cell-surface molecules CD4, CD25 and CD45RB. The percentage of CD25⁺ CD45RBIo of all CD4⁺ T-lymphocytes in lung tissue or lung-draining lymph nodes was determined by flow cytometry. The number of CD4⁺ CD25⁺ CD45RB^{lo} cells in lung tissue from ovalbumin challenged mice (n=3) was significantly (P<0.01, student's t-test) increased as compared to the number in lung tissue from control mice (n=4) challenged with saline (21.75±0.63 versus 9.25±0.93, respectively). In contrast, the number of CD4⁺ CD25⁺ CD45RB^{lo} cells in lung-draining lymph nodes were not different between ovalbumin and saline-challenged mice (6.19±1.21 versus 5.49±1.01, respectively).

These data clearly demonstrate that upon allergen inhalation by previously sensitized mice, the number of professional Treg cells (CD4⁺ CD25⁺ CD45RB^{lo}) increases specifically in the affected organ, the lung.

### EXAMPLE 2

Inactivation of Treg Cells by an Activating Monoclonal Antibody to GITR Potentiates Allergen-Induced Airway Manifestations of Asthma

We examined whether inactivation of professional Treg cells by an activating antibody to GITR was able to affect the induction of airway manifestations of asthma in a mouse model of allergic asthma. Balb/c mice were sensitized by ovalbumin in alum adjuvant as described previously (Deurloo et al., 2001). Twenty days after sensitization, blood samples were obtained to determine serum IgE antibody levels to ovalbumin. Subsequently, the airway responsiveness to the bronchospasmogenic stimulus methacholine was determined by whole-body plethysmography as described previously (Deurloo et al., 2001). Prior to the first ovalbumin inhalation challenge, mice were divided into two groups of 6 animals: one group received an intraperitoneal injection 1 mg of endotoxin-free monoclonal antibody to GITR (DTA-1); the other group received 1 mg rat IgG as control treatment. Subsequently, mice were challenged three times (once a day, every third day) by inhalation of ovalbumin (10 mg/ml). Twenty-four hours after the last inhalation challenge, airway responsiveness to methacholine was again determined (Deurloo et al., 2001).

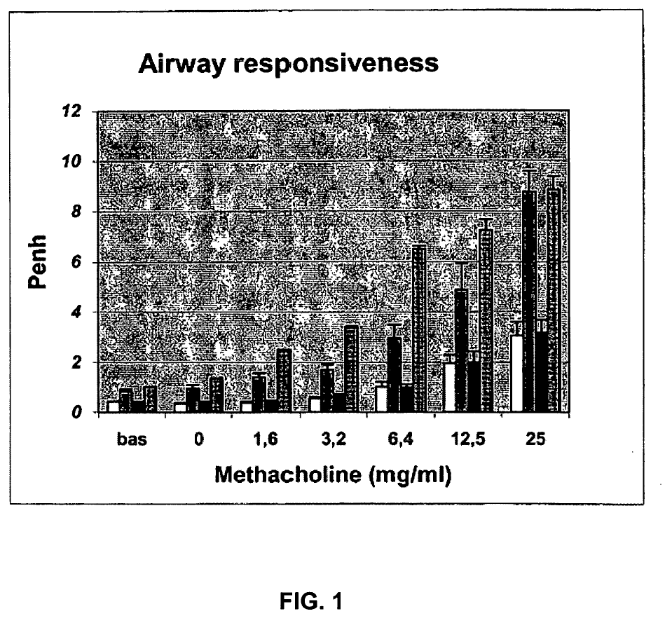

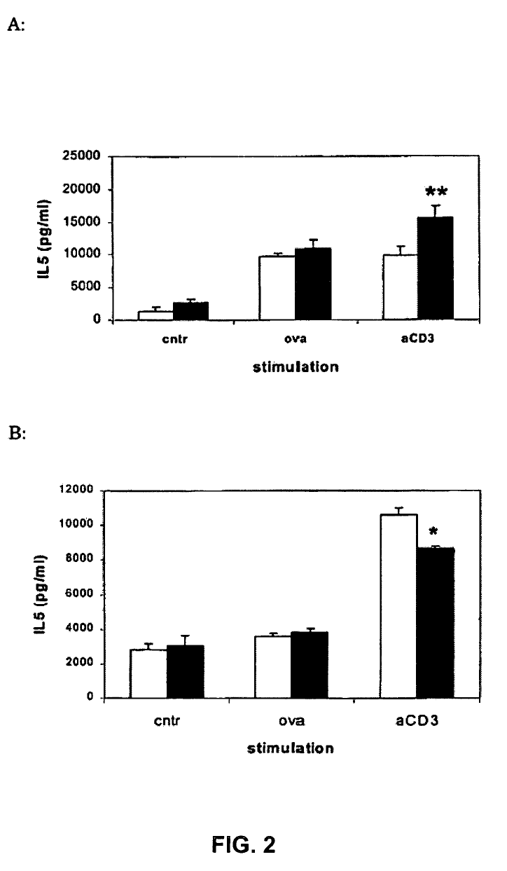

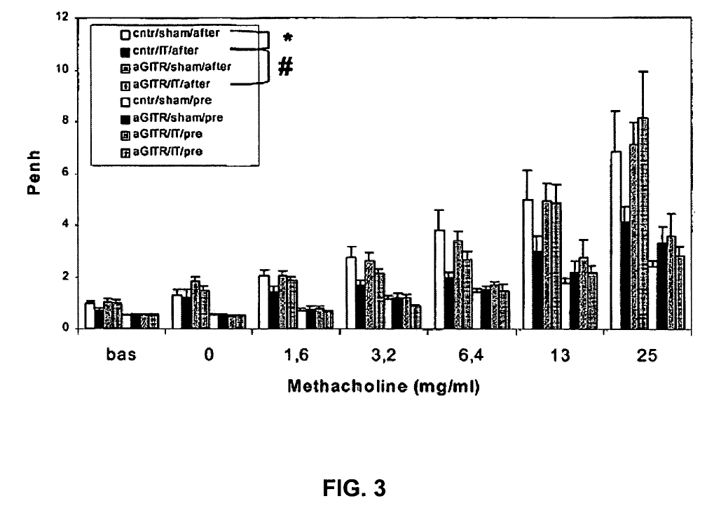

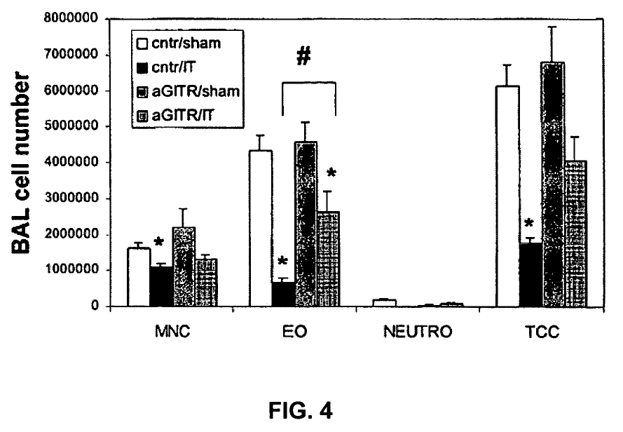

In mice treated with control antibody, serum levels of ovalbumin specific IgE were significantly increased after ovalbumin challenge as compared to pre-challenge values (Table 1). Interestingly, treatment with anti-GITR strongly potentiated (P<0.01, student's t-test) the upregulation of serum ovalbumin-specific IgE levels after ovalbumin inhalation challenge (Table 1). Furthermore, in mice treated with treated with control antibody, the airway responsiveness to methacholine was significantly increased after ovalbumin challenge as compared to pre-challenge values (FIG. 1). Treatment with anti-GITR strongly potentiated (P<0.01) the allergen-induced airway hyperresponsiveness to methacholine (FIG. 1). Moreover, a leftward shift of the dose-response curve, a cardinal feature of severe asthma, was observed in ovalbumin-challenged mice treated with anti-GITR as compared to control antibody treated mice. The ED₅₀ value of the methacholine dose-response curve in ovalbumin challenged mice was significantly (P<0.01) decreased from 12.6±1.5 mg/ml methacholine after treatment with control antibody to 5.5±1.3 mg/ml in mice treated with anti-GITR.

It is concluded that inactivation of naturally occurring professional Treg cells by treatment with an activating antibody to GITR strongly potentiates allergen induced airway hyperresponsiveness and IgE upregulation in this mouse model of asthma.

Table 1. Serum levels of ovalbumin-specific IgE (Units/ml). Ovalbumin sensitized BALB/c mice (n=6 per group) were treated with control antibody (Control) or anti-GITR prior to repeated ovalbumin inhalation challenges. Levels of ovalbumin-specific IgE in serum were measured before and after ovalbumin inhalation challenges. *: P<0.01 as determined by student's t-test and compared to the levels before ovalbumin inhalation challenge. #: P<0.01 as determined by student's t-test and compared to control antibody treated mice.

| Treatment | Before | After |
|---|---|---|
| Control | 4,988 ± 1,788 | 87,581 ± 24,189* |
| Anti-GITR | 7,373 ± 1,806 | 321,380 ± 94,270*# |

### EXAMPLE 3

Inactivation of Treg Cells by an Activating Monoclonal Antibody to GITR Potentiates Th2 Type Cytokine Production.

Next, we examined the cytokine production upon anti-CD3 restimulation of lung lymphocytes obtained from ovalbumin sensitized and challenged mice treated in vivo with control antibody or anti-GITR (see example 2). Lymphocytes were isolated from ovalbumin sensitized and challenged mice and restimulated in vitro as described previously (Deurloo et al., 2001). Lung lymphocyte cultures from animals treated in vivo with anti-GITR during antigen inhalation challenge, produced significantly (P<0.05, student's t-test) potentiated amounts of the Th2 type cytokines IL5, IL10 and IL13 as compared to mice treated with control antibody (Table 2).

It is concluded that inactivation of naturally occurring professional Treg 5 cells by treatment with an activating antibody to GITR potentiates Th2 type cytokine production.

Table 2. Cytokine production by lung lymphocytes obtained from ovalbumin sensitized and challenged BALB/c mice treated in vivo with control antibody (control) or anti-GITR. Lung lymphocyte cultures were restimulated with plate-bound anti-CD3 for five days. *: P<0.05 and **: P<0.01 as determined by student's t-test and compared to control antibody treated mice.

| Treatment | IL5 (pg/ml) | IL10 (pg/ml) | IL13 (pg/ml) |
|---|---|---|---|
| Control | 15,872 ± 6,451 | 285 ± 37 | 2,942 ± 736 |
| Anti-GITR | 39,100 ± 4,346* | 1,046 ± 226** | 11,653 ± 1,670** |

### Experimental Part 2

### Materials and Methods

Animals were sensitized and challenged and subsequently, lung cells were isolated and re-stimulated as described above. Lung cell cultures were re-stimulated with plate-bound anti-CD3 or with ovalbumin in the presence of 10 μg/ml anti-GITR (clone DTA-1) or rat IgG as control or 1 μg/ml recombinant mouse GITR-Fc fusion protein (Komed, Seoul, Korea) or human IgG as control. Each stimulation was performed in triplicate. After 5 days of culture at 37° C., the supernatants were harvested, pooled per stimulation, and stored at −20° C. until IL-5 levels were determined by ELISA. IL-5 ELISA was performed according to the manufacturers' instructions (BD Pharmingen) with a detection limit of 32 pg IL-5 per ml.

### Results

We determined whether a GITR activating antibody is able to affect cytokine production of lung lymphocytes from sensitized mice isolated 24 hours after OVA inhalation. Lung lymphocyte cultures stimulated with anti-CD3 produced significantly (P<0.01) higher levels of the prototypic Th2 cytokine IL-5 in the presence of anti-GITR than in the presence of control antibody (FIG. 2A). In contrast, IL-5 production upon ovalbumin restimulation was not affected by anti-GITR. Thus, functional inactivation of Treg cells by treatment with anti-GITR only potentiates IL-5 production by lung lymphocytes when they are activated through their T-cell receptor in vitro. Collectively, these data demonstrate that natural Treg cells are present in allergen-challenged lungs and can actively suppress IL-5 production.

Next, we examined whether blocking of GITR:GITRL interaction on natural Treg cells by recombinant mouse GITR-Fc fusion protein, which selectively binds to GITRL and thereby prevents activation of GITR, is able to inhibit Th2 responses in vitro. Lung lymphocyte cultures incubated with GITR-Fc produced significantly (P<0.05) less IL-5 upon anti-CD3 restimulation than control cultures incubated with human IgG (FIG. 2B). This inhibition was not observed in lung lymphocyte cultures re-stimulated with ovalbumin or in non-stimulated cultures.

It is concluded that naturally occurring professional Treg cells, which are present in lung lymphocyte cultures, suppress the activation and cytokine production of Th2 cells when they are activated by anti-CD3. Interestingly, the suppression of Th2 activation and IL-5 production after anti-CD3 stimulation can be potentiated by blocking of GITR:GITRL interaction using GITR-Fc. Therefore, GITR-Fc is one of the compounds that is able to prevent activation of GITR by its natural ligand, GITRL, and is useful to potentiate the suppressive functions of naturally occurring professional Treg cells. GITIR-Fc is one of the compounds that can be used for the treatment of allergic disorders as described in this patent.

### Experimental Part 3

Natural Treg cells may be important for the induction of adaptive Treg cells, either directly or through an effect on antigen-presenting cells. We have demonstrated that allergen-immunotherapy induces a suppressive memory response mediated by the immunosuppressive cytokine IL-10 in a mouse model. Here we examine whether functionally active natural Treg cells are essential for the induction of this IL-10 dependent suppressive memory response.

### Materials and Methods

Animals. Animal care and use were performed in accordance with the guidelines of the Dutch Committee of Animal Experiments. Specific pathogen-free male BALB/c mice (6-8 wk old) were purchased from Charles River (Maastricht, NL) and housed in macrolon cages in a laminar flow cabinet and provided with food and water ad libitum.

Sensitization, treatment and challenge. Mice were sensitized intraperitoneally (i.p.) on days 0 and 7 with 10 μg ovalbumin (OVA, grade V, Sigma-Aldrich) in 0.1 ml alum (Pierce, Rockford, Ill.). Two weeks after the last sensitization, the mice were divided in four groups. Thirty minutes prior to immunotherapy, mice were injected with 0.5 mg rat monoclonal antibody to mouse GITR (DTA-1) or control antibody (rat IgG). Sham-immunotherapy and OVA-immunotherapy was carried out with 3 s.c. injections of respectively 0.2 ml pyrogen-free saline (B. Braun, Melsungen, Del.) or 1 mg OVA in 0.2 ml pyrogen-free saline on alternate days. Five weeks after treatment, when the antibodies were cleared from the blood, mice were exposed to three OVA inhalation challenges (10 mg/ml in saline) for 20 min every third day.

Measurement of airway responsiveness in vivo. Airway responsiveness to methacholine was measured after treatment but before OVA challenge (pre measurement) and twenty-four hours after the last OVA challenge. Airway responsiveness was measured in conscious, unrestrained mice using barometric whole-body plethysmography by recording respiratory pressure curves (Buxco, EMKA Technologies, Paris, FR) in response to inhaled methacholine (acetyl-p-methylcholine chloride, Sigma-Aldrich). Airway responsiveness was expressed in enhanced pause (Penh), as described in detail previously (Deurloo et al., 2001). Briefly, mice were placed in a whole-body chamber and basal readings were determined for 3 min. Aerosolized saline, followed by doubling concentrations of methacholine (ranging from 1.6-25 mg/ml in saline), were nebulized for 3 min, and readings were determined for 3 min after each nebulization.

Analysis of the cellular composition of the bronchoalveolar lavage fluid. Bronchoalveolar lavage (BAL) was performed immediately after bleeding of the mice by lavage of the airways through a tracheal cannula 5 times with 1 ml saline (37° C.). Cells in the BALF were centrifuged and resuspended in cold PBS. The total number of cells in the BALF was determined using a Bürker-Türk counting-chamber (Karl Hecht Assistant KG, Sondheim/Röhm, DE). For differential BALF cell counts, cytospin preparations were made (15×g, 5 min, 4° C., Kendro Heraues Instruments, Asheville, N.C.). Next, cells were fixed and stained with Diff-Quick (Dade A. G., Dudingen, CH). Per cytospin, 200 cells were counted and differentiated into mononuclear cells, eosinophils, and neutrophils by standard morphology and staining characteristics.

Statistical analysis. All data are expressed as mean±standard error of mean (SEM). The airway dose-response curves to methacholine were statistically analyzed by a general linear model of repeated measurements followed by post-hoc comparison between groups. Data were log transformed before analysis to equalize variances in all groups. Statistical analysis on BALF cell counts was performed using Student's t test (2-tailed, homosedastic). Results were considered statistically significant at the p<0.05 level.

### Results

Airway responsiveness in vivo. No significant differences between all four groups were observed in airway responsiveness to methacholine prior to OVA challenge (FIG. 3). After OVA-inhalation challenge, all mice display significant airway hyper responsiveness to methacholine as compared to before challenge. However, OVA-immunotherapy significantly reduced (P<0.05) AHR to methacholine as compared to sham-treated mice. Interestingly, treatment with anti-GITR completely prevented the reduction of AHR to methacholine after OVA-immunotherapy. It can be concluded that natural Treg cells are essential for the induction of the beneficial effect of OVA-immunotherapy on AHR.

Cellular composition of the bronchoalveolar lavage fluid. In BALB/c mice, no eosinophils are present in BALF prior to OVA inhalation. OVA sensitized BALB/c mice that received sham-immunotherapy showed BALF eosinophilia after OVA inhalation challenge (FIG. 4). After OVA challenge of mice that received OVA-immunotherapy, the number of BALF eosinophils was significantly reduced as compared to sham-treated mice. Treatment with anti-GITR significantly reversed the reduction of BALF eosinophilia after OVA-immunotherapy. It can be concluded that natural Treg cells are essential for induction of the beneficial effect of OVA-immunotherapy on airway eosinophilia.

Altogether, it can be concluded that, at the time of immunotherapy, natural Treg cells are essential for the induction of a regulatory response that is able to inhibit allergen-induced airway manifestations of asthma. This implicates that allergen immunotherapy may only be effective in allergic patients that have sufficient numbers of functionally active natural Treg cells. Therefore, measurement of the number of Tregs cells in the blood of allergic patients by specific markers, including but not limited to CD25, CTLA4, CD45RO, GITR and FoxP3, will be an important diagnostic tool to determine whether it is worthwhile to start allergen-immunotherapy. Moreover, methods to increase the number or function of natural Treg cells in allergic patients prior to or concomitant with allergen immunotherapy will improve the beneficial effects of immunotherapy.

### REFERENCES

Bystry, R. S., Aluvihare, V., Welch, K. A., Kallikourdis, M. & Betz, A. G. (2001). B cells and professional APCs recruit regulatory T cells via CCL4. Nat Immunol, 2, 1126-32.
Caramalho, I., Lopes-Carvalho, T., Ostler, D., Zelenay, S., Haury, M. & Demengeot, J. (2003). Regulatory T cells selectively express Toll-like receptors and are activated by lipopolysaccharide. J Exp Med, 197, 403-11.
Colantonio, L., lellem, A., Sinigaglia, F. & D'Ambrosio, D. (2002). Skin-homing CLA+T cells and regulatory CD25⁺ T cells represent major subsets of human peripheral blood memory T cells migrating in response to CCL1/I-309. Eur J Immunol, 32, 3506-14.
Deurloo, D. T., van Esch, B. C., Hofstra, C. L., Nijkamp, F. P. & van Oosterhout, A. J. (2001). CTLA4-IgG reverses asthma manifestations in a mild but not in a more “severe” ongoing murine model. Am J Respir Cell Mol Biol, 25, 751-60.
Dieckmann, D., Bruett, C. H., Ploettner, H., Lutz, M. B. & Schuler, G. (2002). Human CD4(+)CD25(+) Regulatory, Contact-dependent T Cells Induce Interleukin 10-producing, Contact-independent Type 1-like Regulatory T Cells. J Exp Med, 196, 247-53.
Iellem, A., Mariani, M., Lang, R., Recalde, H., Panina-Bordignon, P., Sinigaglia, F. & D'Ambrosio, D. (2001). Unique chemotactic response profile and specific expression of chemokine receptors CCR4 and CCR8 by CD4(+)CD25(+) regulatory t cells. J Exp Med, 194, 847-54.
Jonuleit, H., Schmitt, E., Kakirman, H., Stassen, M., Knop, J. & Enk, A. H. (2002). Infectious Tolerance: Human CD25(+) Regulatory T Cells Convey Suppressor Activity to Conventional CD4(+) T Helper Cells. J Exp Med, 196, 255-60.
Jonuleit, H., Schmitt, E., Schuler, G., Knop, J. & Enk, A. H. (2000). Induction of Interleukin 10-producing, Nonproliferating CD4(+) T Cells with Regulatory Properties by Repetitive Stimulation with Allogeneic Immature Human Dendritic Cells. J Exp Med, 192, 1213-1222.
Jonuleit, H., Schmitt, E., Stassen, M., Tuettenberg, A., Knop, J. & Enk, A. H. (2001a). Identification and functional characterization of human cd4(+)cd25(+) t cells with regulatory properties isolated from peripheral blood. J Exp Med, 193, 1285-94.
McHugh, R. S., Whitters, M. J., Piccirillo, C. A., Young, D. A., Shevach, E. M., Collins, M. & Byrne, M. C. (2002). CD4(+)CD25(+) Immunoregulatory T Cells. Gene expression analysis reveals a functional role for the glucocorticoid-induced TNF receptor. Immunity, 16, 311-23.
Pasare, C. & Medzhitov, R. (2003). Toll Pathway-Dependent Blockade of CD4⁺ CD25+T Cell-Mediated Suppression by Dendritic Cells. Science, 16, 16.
Roncarolo, M. & Levings, M. K. (2000). The role of different subsets of T regulatory cells in controlling autoimmunity. Curr Opin Immunol, 12, 676-83.
Shevach, E. M. (2002). CD4⁺ CD25⁺ suppressor T cells: more questions than answers. Nat Rev Immunol, 2, 389-400.
Shimizu, J., Yamazaki, S., Takahashi, T., Ishida, Y. & Sakaguchi, S. (2002). Stimulation of CD25⁺ CD4⁺ regulatory T cells through GITR breaks immunological self-tolerance. Nat Immunol, 3, 135-142.
Suto, A., Nakajima, H., Kagami, S. I., Suzuki, K., Saito, Y. & Iwamoto, I. (2001). Role of cd4(+) cd25(+) regulatory t cells in t helper 2 cell-mediated allergic inflammation in the airways. Am J Respir Crit Care Med, 164, 680-7.
Thornton, A. M. & Shevach, E. M. (2000). Suppressor effector function of CD4⁺ CD25+immunoregulatory T cells is antigen nonspecific. J Immunol, 164, 183-90.
Zuany-Amorim, C., Sawicka, E., Manlius, C., Le Moine, A., Brunet, L. R., Kemeny, D. M., Bowen, G., Rook, G. & Walker, C. (2002). Suppression of airway eosinophilia by killed Mycobacterium vaccae-induced allergen-specific regulatory T-cells. Nat Med, 8, 625-9.

1. A method for regulating tolerance to an allergen in a subject, said method comprising:
providing, in the allergen's presence, the subject with an inhibitor able to regulate the activation of a glucocorticoid-induced tumor necrosis factor receptor (GITR).

2. A method for inducing and/or increasing tolerance to an allergen in a subject, said method comprising:
providing, in the presence of the allergen, the subject with an inhibitor able to at least in part preventing activation of glucocorticoid-induced tumor necrosis factor receptor (GITR).

3. The method according to claim 2, wherein said inhibitor at least in part prevents ligation of GITR by an endogenous GITR-ligand (GITRL).

4. The method according to claim 2, wherein said GITR is present on a CD4⁺ CD25⁺ regulatory T cell.

5. The method according to claim 2, wherein said inhibitor is an inhibitor of GITR.

6. The method according to claim 2, wherein said inhibitor at least in part inhibits GITR expression.

7. The method according to claim 2, wherein said inhibitor is an inhibitor of a GITRL.

8. The method according to claim 2, wherein said inhibitor at least in part inhibits GITRL expression.

9. The method according to claim 1, wherein the subject is further provided with the allergen.

10. The method according to claim 1, wherein said allergen is an allergen involved in allergic disease.

11. The method according to claim 10, wherein the allergic disease is asthma.

12. The method according to claim 1, further comprising providing the subject with a compound able to activate a Toll-like receptor.

13. The method according to claim 1, further comprising providing the subject with a compound able to inhibit activation of an antigen presenting cell.

14. A method for obtaining a compound capable of at least in part preventing activation of glucocorticoid-induced tumor necrosis factor receptor (GITR), said method comprising the steps of:
incubating a GITR protein or a functional equivalent and/or a functional fragment thereof with a candidate compound;
determining whether said candidate compound binds to said GITR protein or a functional equivalent and/or a functional fragment thereof;
determining whether said candidate compound blocks or mimics an effect mediated by a GITR-GITR-ligand (GITRL) interaction; and
selecting a compound that blocks an effect mediated by a GITR-GITRL interaction, thus obtaining a compound capable of at least in part preventing activation of GITR.

15. The method according to claim 14, further comprising testing said compound in a non-human animal with features reminiscent of an allergic disease.

16. An isolated, recombinant or synthetic compound obtainable by the method of claim 14.

17. The isolated, recombinant or synthetic compound of claim 16, wherein said compound is a proteinaceous substance.

18. The isolated, recombinant or synthetic compound of claim 17, wherein said proteinaceous substance is an antibody and/or a functional fragment thereof.

19. An isolated nucleic acid encoding the isolated, recombinant or synthetic compound of claim 17.

20. A vector comprising the nucleic acid of claim 19.

21. A gene delivery vehicle comprising the vector of claim 20.

22. A pharmaceutical composition comprising:
the isolated, recombinant or synthetic compound of claim 16.

23. The pharmaceutical composition of claim 22, further comprising an allergen.

24. The pharmaceutical composition of claim 23, wherein said allergen is an allergen involved in allergic disease.

25. The pharmaceutical composition of claim 24, wherein the allergic disease is asthma.

26. A method of treating an allergy in a subject, the method comprising:
administering to the subject the pharmaceutical composition of claim 22.

27. The method according to claim 26 wherein said compound is an inhibitor of GITR or wherein said compound at least in part inhibits the expression of GITR or wherein said compound is an inhibitor of a GITRL or wherein said compound at least in part inhibits the expression of GITRL.

28. The method according to claim 1, further comprising providing the subject with a compound capable of activating a CTLA4 receptor.

29. A method for suppressing glucocorticoid-induced tumor necrosis factor receptor (GITR) activation of cells expressing GITR, said method comprising:
providing, in the presence of an allergen, said cells with an inhibitor of GITR.

30. The method according to claim 29, wherein said inhibitor of GITR comprises GITR-Fc.

31. A method for selecting an allergic individual responsive to allergen-immune treatment, said method comprising:
measuring whether the allergic individual's number of CD4⁺ CD25⁺ Treg cells in the allergic individual's blood is sufficient to start allergen-immune treatment.

32. A method for determining responsiveness of an allergic individual to allergen-immune treatment, said method comprising:
measuring whether the allergic individual's number of CD4⁺ CD25⁺ Treg cells in the allergic individual's blood increases in response to allergen-immune treatment.

33. A method of improving tolerance to an allergen in an individual, said method comprising:
increasing the individual's number and/or function of natural Treg cells.

34. The method according to claim 33, wherein said increasing is prior to, or concomitant with providing the allergen.

35. The method according to claim 33, wherein said increasing is caused by contacting T-cells with TGFbeta.
